(19) United States
(12) Patent Application Publication
(10) Pub. No.: US 2024/0016417 A1
(43) Pub. Date: Jan. 18, 2024

(54) METHOD AND SYSTEM FOR DETECTION OF GLUCOSE AND OTHER COMPOUNDS USING SWIRR

(71) Applicant: TriEye Ltd., Tel Aviv (IL)

(72) Inventors: Erga Lifshitz, Tel Aviv (IL); Elior Dekel, Tel Aviv (IL); Dan Kuzmin, Tel Aviv (IL); Ariel Danan, Tel Aviv (IL); Avraham Bakal, Tel Aviv (IL); Uriel Levy, Tel Aviv (IL); Omer Kapach, Tel Aviv (IL)

(21) Appl. No.: 18/255,137

(22) PCT Filed: Dec. 7, 2021
(86) PCT No.: PCT/IB2021/061425

§ 371 Date: May 31, 2023

Related U.S. Application Data
(60) Provisional application No. 63/122,159, filed on Dec. 7, 2020.

(51) Int. Cl. A61B 5/145; A61B 5/1455

(52) U.S. Cl. CPC A61B 5/14532; A61B 5/1455; A61B 5/6824

(57) ABSTRACT

A device and method for detecting the level of a compound in tissue, the device including: an illumination source operable to emit light from an optical opening into the tissue; a detector array having a plurality of photosites, each operable to detect light of the illumination source travelling through the tissue; wherein different photosites of the detector array are located at different distances from the optical opening; and a processor adapted for determining compound levels in the tissue based on differences in detected illumination levels at distinct wavelengths at different distances from the optical opening.

### CROSS REFERENCE TO RELATED APPLICATIONS

This is a 371 application from international patent application PCT/IB2021/061425 filed Dec. 7, 2021, which claims priority from U.S. provisional patent application Ser. No. 63/122,159 filed Dec. 7, 2020 which is incorporated herein by reference in its entirety.

### BACKGROUND

Glucose levels in blood are essential indicators for diabetes and other health conditions and their regular monitoring plays a large part in treating these conditions. Therefore, devices for noninvasive, infection free, and safe monitoring of blood glucose levels are of particular interest.

Normal glucose levels in blood for a non-diabetic person are, according to some accepted measures, 70 to 99 mg/dL (milligrams per deciliter) after fasting, and less than 140 mg/dL one to two hours after eating. For diabetics (individuals suffering from diabetes), normal blood sugar levels are considered between 80 to 130 mg/dL after fasting, and less than 180 mg/dL one to two hours after eating. According to FDA regulations, a device providing self-monitoring of blood glucose should have about ±15 mg/dl error relative to a reference value.

One method for glucose measurement proposes the use of near infrared (NIR) diffuse-reflectance spectroscopy (DRS) where a spectroscopic measurement is taken from a reflection from the from the upper skin layers and the glucose level is calculated from the divergence of the spectral response. While NIR DRS shows promise for blood glucose monitoring, measurement errors reported by studies with this method are of the order of 16-30 mg/dl which is not sufficiently low to meet standard FDA regulations.

There is therefore a need in the art for an effective noninvasive glucose concentration sensor that provides the required sensitivity.

### SUMMARY

This disclosure describes systems and methods for noninvasive glucose concentration measurements, as well as noninvasive measurement of other materials. Using a detector array operating in the short-wave infrared (SWIR) band of the electromagnetic spectrum enables DRS with the required accuracy. Use of a detector including an array of photosites compared to a single photosite detector may provide for multiple instantaneous measurements and several statistical advantages including (but not limited to):
Statistically better SNR by averaging signals from photosites located at similar distances to the illumination source.
Immediate exclusion of irregular measurement points (moles, veins, etc.) by using image processing of the signal received from the multiple photosites.
The intensity to radius dependency can be calibrated and adds another parameter along with the spectral response.

SWIR is well suited for noninvasive glucose concentration measurements for several reasons, such as (but not limited to):
Blood absorption in the SWIR range changes significantly with changes in glucose concentration levels. For example, FIG. 5 is a chart (taken from “In Vivo Noninvasive Measurement of Blood Glucose by Near-Infrared Diffuse-Reflectance Spectroscopy”, Maruo et al.) showing in-vivo glucose spectral regression coefficients in SWIR.
The clear absorption lines around 950 nm, 1200 nm, and 1400 nm provide for high contrast measurements.
Tissue is very transmissive in the SWIR range, meaning low skin absorption.
SWIR operates at eye-safe wavelengths.

Therefore, the combination of a detector array and use of SWIR wavelengths may provide the required accuracy for noninvasive glucose concentration measurement. In some embodiments, the system described herein may be adapted for detection in tissue of levels of other compounds, such as but not limited to cholesterol or hemoglobin.

According to some aspects, a device for detecting compound levels in tissue includes: an illumination source operable to emit light from an optical opening into the tissue; a detector array having a plurality of photosites, each photosite operable to detect light of the illumination source travelling through the tissue, wherein different photosites of the detector array are located at different distances from the optical opening; and a processor configured for determining the compound levels in the tissue based on differences in detected illumination levels at distinct wavelengths at different distances from the optical opening.

In some embodiments, the compound is glucose, wherein the light emitted from the illumination source has distinct wavelengths, wherein the distinct wavelengths are those where glucose is absorptive, and wherein the processor is adapted to determine glucose levels in the tissue based on the amount of light received by the detector array at each of the distinct wavelengths. In some embodiments, the compound is hemoglobin, wherein the light emitted from the illumination source has distinct wavelengths, wherein the distinct wavelengths are those where hemoglobin is absorptive, and wherein the processor is configured to determine hemoglobin levels in the tissue based on the amount of light received by the detector array at each of the distinct wavelengths.

In some embodiments, the detector array includes a color filter array (CFA) for permitting transmission of the distinct wavelengths to the detector array for measuring of the distinct wavelengths by the detector array. In some embodiments, the CFA permits transmission of non-overlapping wavelengths to different rows or columns of the detector array. In some embodiments, each photosite is adapted to detect one of the distinct wavelengths.

In some embodiments, the processor is configured to calibrate the device by comparing measured compound levels to an illumination transmission model determined for the subject based on an invasive measurement of different glucose concentration levels. In some embodiments, the processor is configured to determine an average of a plurality of detected illumination levels of different photosites whose distance from the illumination source is within a similar distance range.

In some embodiments, the illumination source includes multiple illumination sources positioned in a ring arrangement.

In some further aspects, a method for detecting compound levels in tissue includes: illuminating a surface of the tissue by an illumination source having an optical opening; detecting reflected and/or transmitted light from the illumination source by a photodetector array includes a plurality of photosites, wherein different photosites of the detector array are located at different distances from the optical opening; and determining compound levels in the tissue based on differences in detected illumination levels at distinct wavelengths at different distances from the optical opening by a processor.

In some embodiments, the compound is glucose, wherein the light emitted from the illumination source has distinct wave lengths and wherein the distinct wavelengths are those where glucose is absorptive. In some embodiments, the method further includes determining by the processor of glucose levels in the tissue based on the amount of light received by the detector array at each of the distinct wavelengths.

In some embodiments, the detector array includes a color filter array (CFA) for permitting transmission of the distinct wavelengths to the detector array for measuring of the distinct wavelengths by the detector array. In some embodiments, the CFA permits transmission of non-overlapping wavelengths to each row or column of the detector array.

In some embodiments, each photosite is adapted to detect one of the distinct wavelengths. In some embodiments, the method further includes calibration of the measurement by comparing measured compound levels to an illumination transmission model determined for the subject based on an invasive measurement of different glucose concentration levels.

In some embodiments, the method further includes determining by the processor of an average of a plurality of detected illumination levels of different photosites whose distance from the illumination source is within a similar distance range. In some embodiments, the illumination source includes multiple illumination sources wherein the multiple illumination sources are positioned in a ring arrangement.

In some further aspects, a blood concentration measuring device includes one or more processors and at least one non-transitory computer readable medium having stored thereon instructions that, when executed by the one or more processors, cause the blood concentration measuring device to: illuminate a surface of a tissue with an illumination source having an optical opening; detect reflected and/or transmitted light from the illumination source using a photodetector array includes a plurality of photosites; and determine compound levels in the tissue based on differences in detected illumination levels at distinct wavelengths at different distances from the optical opening, wherein different photosites of the detector array are located at different distances from the optical opening.

In some embodiments, the compound is glucose, wherein the light emitted from the illumination source has distinct wavelengths and wherein the distinct wavelengths are those where glucose is absorptive. In some embodiments, the blood concentration measuring device stores instructions that, when executed by the one or more processors, cause the blood concentration measuring device to determine glucose levels in the tissue based on the amount of light received by the detector array at each of the distinct wavelengths.

In some embodiments, the detector array includes a color filter array (CFA) for permitting transmission of the distinct wavelengths to the detector array for measuring of the distinct wavelengths by the detector array. In some embodiments, the CFA permits transmission of non-overlapping wavelengths in each row or column of the detector array.

In some embodiments, each photosite is adapted to detect one of the distinct wavelengths. In some embodiments, the blood concentration measuring device stores instructions that, when executed by the one or more processors, cause the blood concentration measuring device to determine an average of a plurality of detected illumination levels of different photosites whose distance from the illumination source is within a similar distance range.

In some further aspects a device for detecting the level of a compound in tissue includes: an illumination source operable to emit light from an optical opening into the tissue; a detector array having a plurality of photosites, each operable to detect light of the illumination source travelling through the tissue; wherein different photosites of the detector array are located at different distances from the optical opening; and a processor, adapted for determining compound levels in the tissue based on differences in detected illumination levels at distinct wavelengths at different distances from the optical opening.

In some embodiments, the light emitted from the illumination source has distinct wave lengths. In some embodiments, the distinct wavelengths are those where glucose is absorptive. In some embodiments, the processor is adapted to determine glucose levels in the tissue based on the amount of light received by the detector array at each of the distinct wavelengths. In some embodiments, the distinct wavelengths include a wavelength where glucose is not absorbed. In some embodiments, the detector array includes a color filter array (CFA) for permitting transmission of distinct wavelengths to the detector array for measuring of the distinct wavelengths by the detector array.

In some embodiments, the CFA permits transmission of a distinct wavelength in each row or column of the detector array. In some embodiments, each photosite is adapted to detect a distinct wavelength. In some embodiments, the device further includes a barrier for preventing stray light from reaching the photodetector array. In some embodiments, the processor is adapted to calibrate the device by comparing the measured levels to an illumination transmission model determined for the subject based on an invasive measurement of different glucose concentration levels. In some embodiments, the processor is operable to determine an average of a plurality of measurement levels of different photosites whose distance from the illumination source is within a similar distance range.

In some embodiments, the illumination source includes multiple illumination sources. In some embodiments, the multiple illumination sources are positioned in a ring arrangement. In some embodiments, the illumination source and/or optical opening are positioned at an angle relative to the surface of the detector array

According to other aspects, a method for detecting the level of a compound in tissue includes: illuminating a surface of the tissue by an illumination source having an optical opening; detecting reflected and/or transmitted light from the illumination source by a photodetector array including a plurality of photosites, wherein different photosites of the detector array are located at different distances from the optical opening; and determining compound levels in the tissue based on differences in detected illumination levels at distinct wavelengths at different distances from the optical opening by a processor.

In some embodiments, the light emitted from the illumination source has distinct wavelengths. In some embodiments, the distinct wavelengths are those where glucose is absorptive. In some embodiments, the method further includes determining by the processor of glucose levels in the tissue based on the amount of light received by the detector array at each of the distinct wavelengths. In some embodiments, the distinct wavelengths include a wavelength where glucose is not absorbed. In some embodiments, the detector array includes a color filter array (CFA) for permitting transmission of distinct wavelengths to the detector array for measuring of the distinct wavelengths by the detector array. In some embodiments, the CFA permits transmission of a distinct wavelength in each row or column of the detector array. In some embodiments, each photosite is adapted to detect a distinct wavelength. In some embodiments, a barrier is provided for preventing stray light from reaching the photodetector array. In some embodiments, the method further includes calibration of the measurement by comparing the measured levels to an illumination transmission model determined for the subject based on an invasive measurement of different glucose concentration levels. In some embodiments, the method further includes determining by the processor of an average of a plurality of measurement levels of different photosites whose distance from the illumination source is within a similar distance range.

The details of one or more implementations are set forth in the accompanying drawings and the description below. Other features will be apparent from the description and drawings, and from the claims.

### BRIEF DESCRIPTION OF THE DRAWINGS

Aspects, embodiments and features disclosed herein will become apparent from the following detailed description when considered in conjunction with the accompanying drawings:

FIG. 1 is an illustrative drawing of a device for noninvasive blood concentration measurement consistent with some embodiments of this disclosure;

FIGS. 2A-2G show illustrative drawings of devices for noninvasive blood concentration measurement consistent with some embodiments of this disclosure;

FIG. 3 is an illustrative drawings showing a top view of a BCMD consistent with some embodiments of this disclosure;

FIG. 4 is a diagram of an example process for the operation of a BCMD consistent with some embodiments of this disclosure;

FIG. 5 is a chart showing in-vivo glucose spectral regression coefficients in SWIR.

### DETAILED DESCRIPTION

Reference will now be made in detail to non-limiting examples of this disclosure, examples of which are illustrated in the accompanying drawings. The examples are described below by referring to the drawings, wherein like reference numerals refer to like elements. When like reference numerals are shown, corresponding description(s) are not repeated and the interested reader is referred to the previously discussed figure(s) for a description of the like element(s).

The present disclosure describes technological improvements that enable SWIR based DRS for noninvasive glucose and/or other compound concentration measurements. In some embodiments, DRS uses spectroscopic measurement taken from reflection of SWIR light. Assuming that the distribution of small blood vessels in the skin is uniform, the light absorption in the skin depends only on the path length of the light passing through the skin.

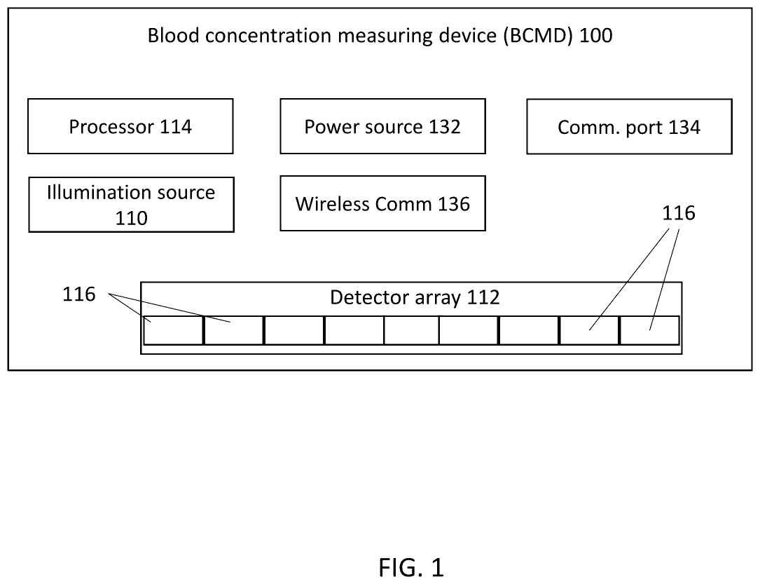

FIG. 1 is an illustrative drawing of a device for noninvasive blood concentration measurement consistent with some embodiments of this disclosure. As shown in FIG. 1, a blood concentration measuring device (BCMD) 100 (or 150 or 160 as described further below) may include one or more illumination sources 110, a detector array 112, and a processor 114. Detector array 112 may include an array of photosites 116. In some embodiments, BCMD 100 may further include a power source 132, a communication port 134, and a wireless communication unit 136.

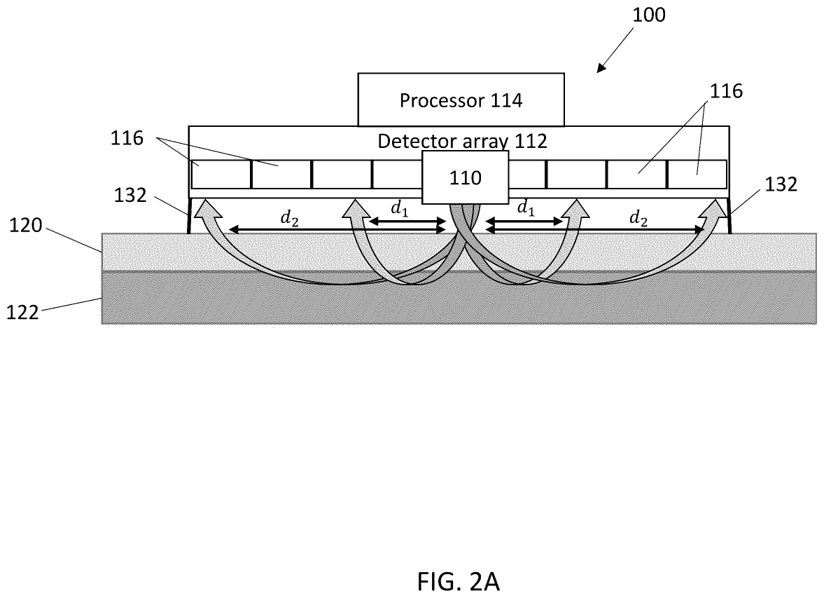

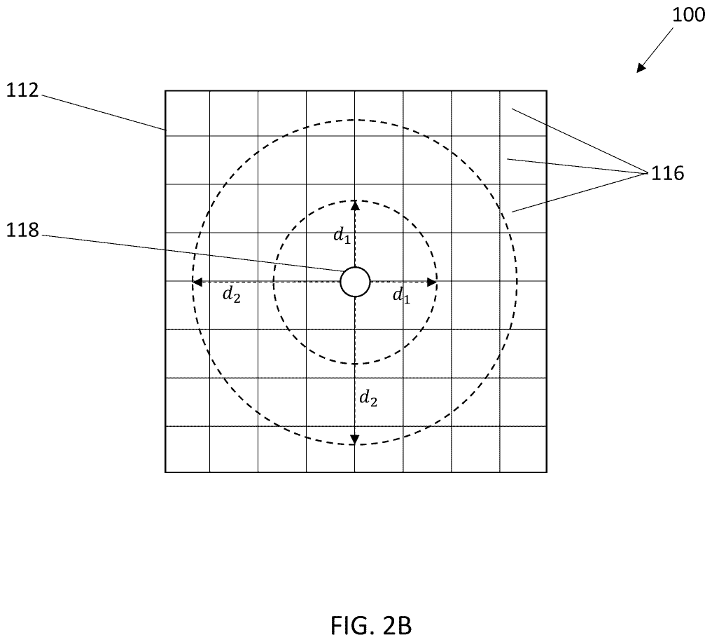

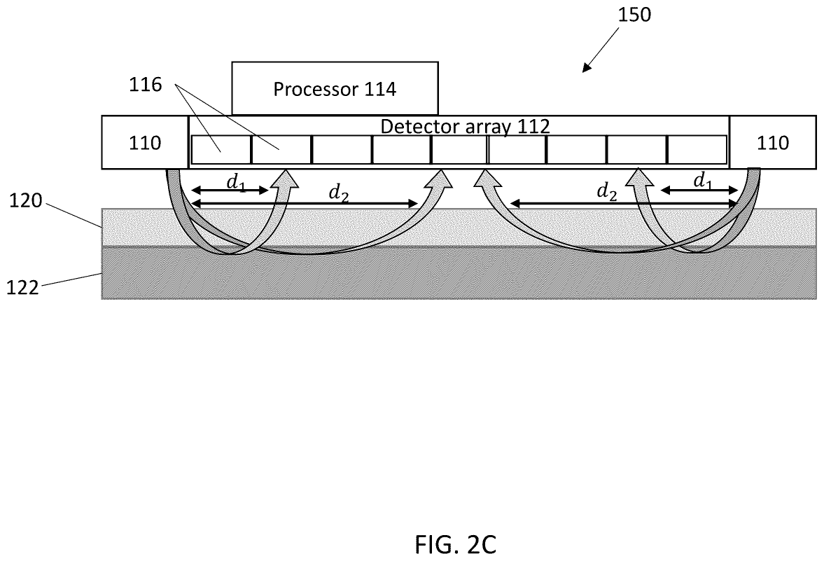

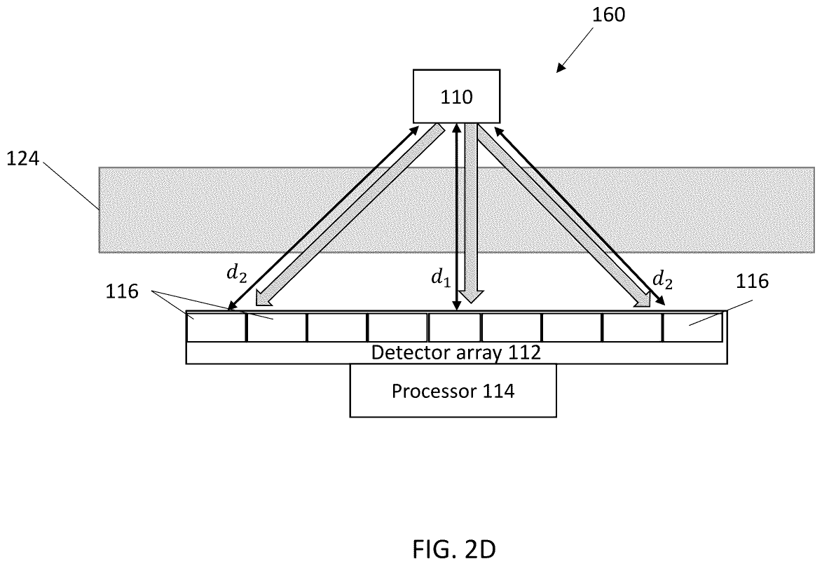

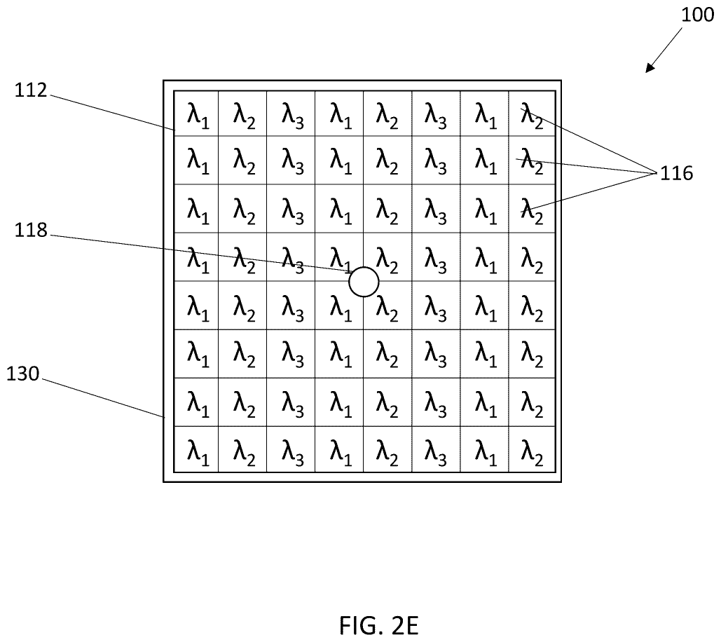

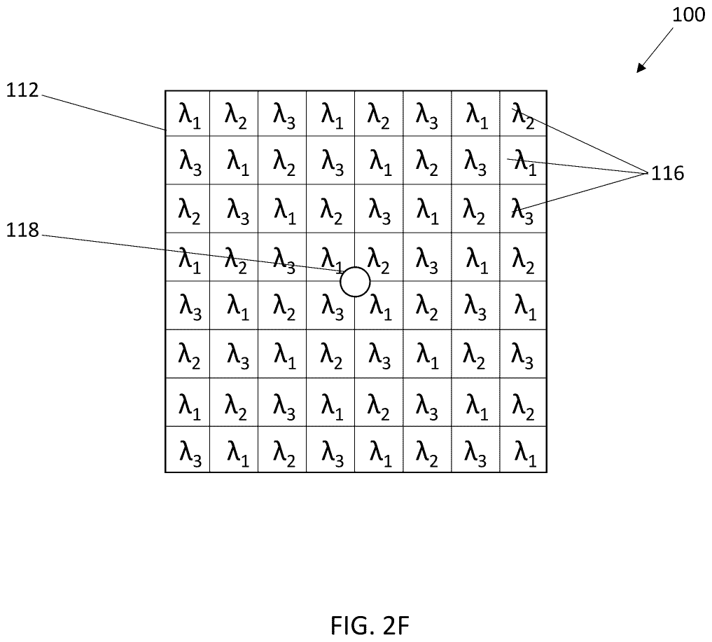

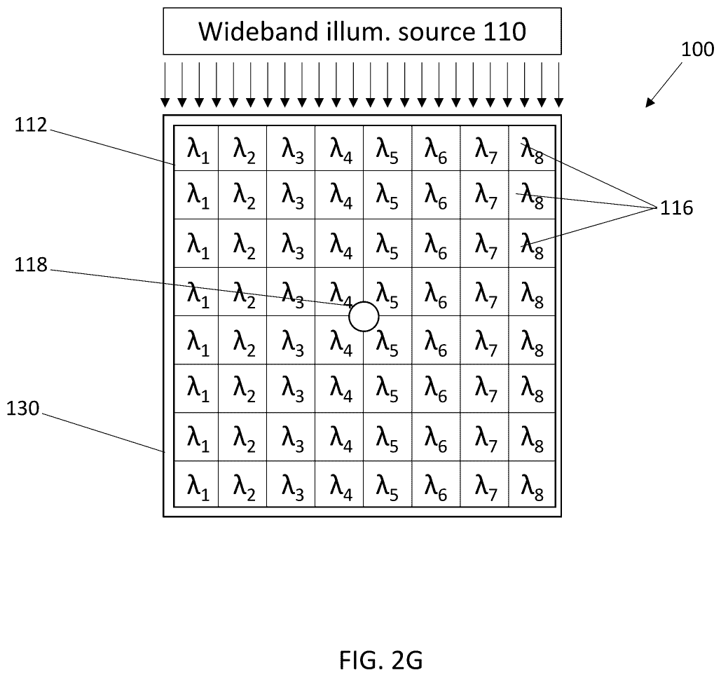

FIGS. 2A-2F show illustrative drawings of devices for noninvasive blood concentration measurement consistent with some embodiments of this disclosure. As shown in FIGS. 2A- 2C BCMD 100 or 150 or 160 may include one or more illumination sources 110, a detector array 112, and a processor 114. Light emitted from illumination sources 110 may pass through epidermal layer 120 and may penetrate partially into dermal layer 122 of the skin before being reflected off dermal layer 122 and the small blood vessels therein.

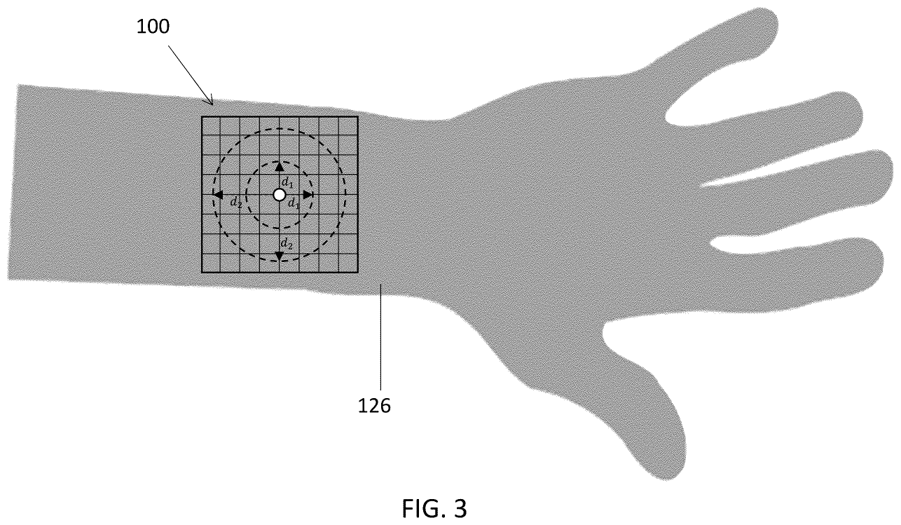

In some embodiments, illumination source 110 may be any one of a point source, or a fiber bundle. Illumination source 110 may include an optical opening 118 (FIGS. 3A, 3B) where the light from illumination source 110 exits illumination source 110 through optical opening 118. In some embodiments, illumination source 110 may provide coherent or incoherent illumination. In some embodiments, illumination source 110 may include one or more LEDs, one or more lasers, or any other suitable source of light. In some embodiments, such as shown in FIG. 2A, BCMD 100 may include an illumination source 110 substantially in the center of detector array 112.

In some embodiments, such as shown in FIG. 2B, BCMD 150 may include multiple illumination sources 110 spread around the detector array 112. FIG. 2B shows two illumination sources 110 but this configuration should not be considered limiting, for example, where device 150 may include multiple illumination sources arranged in a ring shape around detector array 112. In some implementations, each of multiple illumination sources 110 may include one or more LEDs. In some implementations, a central illumination source such as one or more LEDs may transmit light through to each of multiple illumination sources 110. In some embodiments, an optical axis of illumination source 110 and/or an optical axis of optical opening 118 may be positioned at an angle relative to the surface of detector array 112 to thereby direct light into the tissue at an angle to thereby alter the depth of penetration of the light.

In some embodiments, such as shown in FIG. 2C, BCMD 160 may include an illumination source 110 positioned on one side of an appendage 124, such as but not limited to an earlobe or a finger, and detector array 112 positioned on the other side of appendage 124.

As shown in FIG. 2B, in some embodiments, detector array 112 may include photosites 116, where each photosite 116 includes one or more photodiodes (not shown). In some embodiments, photosites 116 may be arranged in a two-dimensional array, such as a rectangular array including at least two columns and two rows of photosites 116, a hexagonal array of photosites 116, and so on.

Each photosite 116 is at a distance (denoted “d”) from optical opening 118 of the one or more illumination sources 110. FIGS. 2A-2D show only two distances d1 and d2 between optical opening 118 and two photosites 116 for simplicity, but it should be appreciated that each photosite 116 in the detector array 112 and optical opening 118 are spaced at a different distance “d” from one another and there are thus multiple distances “d”. In some implementations, the received signal levels at all photosites 116 that are at substantially the same distance from optical opening 118 may be compared and/or averaged. Comparison of detected illumination levels between photosites of approximately the same distance may be used to detect defective photosites and/or bodily irregularities in the inspected tissue. Averaging of the detection signals may be implemented in order to improve signal-to-noise ratio (SNR) of the detection.

It should be appreciated that spreading of detector array 112 over a greater surface area of skin may result in a more accurate measurement of the compound of interest in the tissue due to multiple detection points (photosites 116) per distance from illumination source 110. Since the received signal gets lower as the distance of photodetectors 116 from illumination source 110 increases, the size of detector array 112 is limited by the minimal acceptable SNR. In some embodiments, detector array 112 may have dimensions of up to 1 cm×1 cm. Other physical dimensions may also be implemented.

Processor 114 is a computing device as defined herein. Processor 114 may control the operation of BCMDs 100, 150, 160. Processor 114 may perform operations to determine the concentration of a compound in tissue based on the received levels of light as indicated by detector array 112. Actions said herein to be performed by BCMDs 100, 150, 160, may be understood as being performed by processor 114 including a machine-readable medium that receives machine instructions as a machine-readable signal for instructing or interacting with the components of BCMDs 100, 150, 160.

In some embodiments, a multi-spectral measurement of reflectance or transmittance may be performed in the distinct SWIR wavelengths where glucose is most absorptive, such as: 950 nm, 1150 nm and 1400 nm. In some embodiments, wavelengths corresponding to the absorptive bands of another compound may be measured. It should be appreciated that multiple wavelength measurements allow for higher accuracy of the measurement by comparison of the spectral response for each wavelength separately and collectively.

In some embodiments, a wavelength that is not absorbed by the compound (e.g., glucose, hemoglobin) may be used as one of the measured wavelengths (e.g., using a dedicated illumination source 110 or a dedicated photosite/filter). Signal levels in such a wavelength, if utilized, may be measured by detector 112 for reference and calibration. In some embodiments, illumination source 110 emits substantially white light and detector 112 is adapted for detecting a wavelength that is not absorbed by the compound to be measured for reference and calibration. Since the chosen wavelength is not absorbed by the compound to be measured, the receive levels at the detector should be within an expected range, allowing reference and calibration of detector array 112.

Varying combinations of illumination sources 110 and detector arrays 112 are contemplated for analysis of different wavelengths, such as but not limited to:

In some embodiments, illumination source 110 may be a substantially white light source with each photosite 116 adapted for detecting a specific wavelength such as by using photodiodes that detect a specific wavelength. Non-limiting arrangements of photosites 116 adapted to detect different wavelengths are shown in FIG. 2E, 2F and 2G. As shown in FIG. 2E, each column (or row) may be include photosites 116 for detecting a specific wavelength. As shown in FIG. 2F, each photosite 116 is adapted for detecting a specific wavelength with photosites 116 arranged in a repeating pattern.

FIG. 2G illustrates an example in which a linear filter is placed on top of a broadband photosites array (in which each photosite is capable of detecting any wavelength between λ1 and λ8). Using this method, for each column in the array (in the illustrated example; or another subset of photosites in other implementations) only light which is within the spectral band of λi±Δλ impinges on the photosites and is therefore detected. This allows for an easy and relatively inexpensive way of detecting a plurality of wavelengths over different distances, using a SWIR detector array, such as the ones developed by TriEye, Israel.

In some embodiments, illumination source 110 may be a substantially white light source, with detector array 112 adapted for detecting a specific wavelength such as by covering detector array 112 with a color filter array (CFA) 130 (FIG. 2E). Non-limiting arrangements of photosites 116 adapted to detect different wavelengths by covering with a CFA 130 are shown in FIG. 2E and 2F. Although a CFA is only illustrated in FIG. 2E, it should be appreciated that the pattern of FIG. 2F may be implemented using a CFA. As shown in FIG. 2E, CFA 130 may cover each column (or row) for permitting transmission of non-overlapping wavelengths to each column (or row) enabling detection of a specific wavelength at each photosite 116. As shown in FIG. 2F, a CFA may cover photosites 116 in a repeating pattern. In some embodiments, illumination sources 110 and detector array 112 are implemented using a combination of two or more of the embodiments as described with reference to FIGS. 2B, 2E, 2F, 2G.

In some embodiments, illumination source 110 may include light emitting components for distinct wavelengths of interest and alternately and separately illuminate the emitters of each wavelength. It is noted that whenever distinct wavelengths are mentioned in this disclosure, distinct wavebands (e.g., narrow ones, e.g. spanning 1-20 nm, or wider ones) may be used instead.

In some embodiments (FIG. 2A), BCMD 100 may include a light barrier 132 for preventing the entry of stray light into detector array 112 when BCMD 100 is positioned on the subject for measurement. Stray light may include one or more of stray light of illumination source 110 reflected from other surfaces, light not emitted by illumination source 110, and/or light that has not passed through the tissue of the subject. Embodiments 150 and 160 may also include light barrier 132.

FIG. 3 is an illustrative drawing showing a top view of a BCMD consistent with some embodiments of this disclosure. In the embodiment of FIG. 3, illumination source 110 is positioned at the center of detector array 112 such as in BCMD 100. As shown, multiple photosites 116 may be positioned at distances d from illumination source 110. FIG. 3 shows two distances “d1” and “d2”, but it should be appreciated that multiple distances d may exist between each photosite 116 and optical opening 118 of illumination source 110. FIG. 3 shows detector array 112 placed over a wrist of a subject such as for measurement of the concentration of a compound in the tissue of the subject.

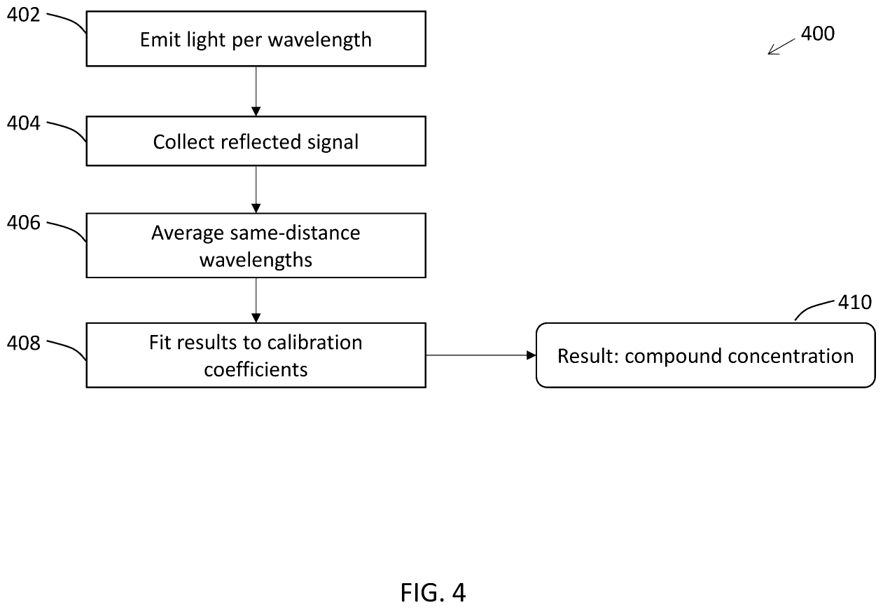

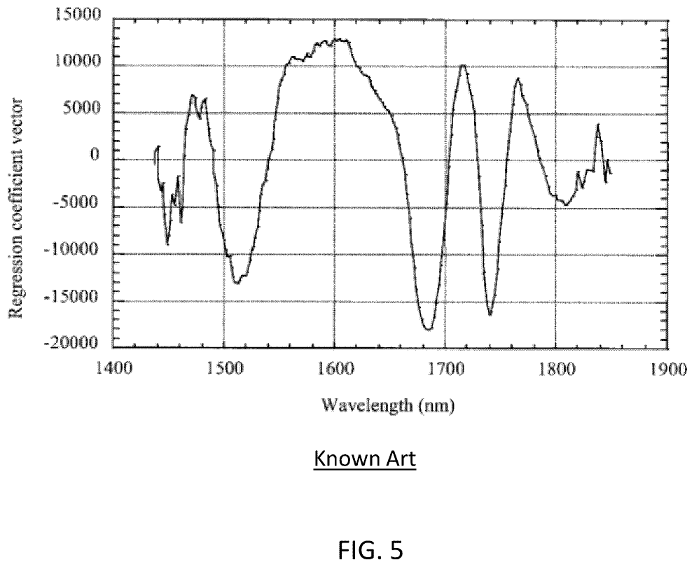

FIG. 4 is a diagram of an example process for the operation of a BCMD consistent with some embodiments of this disclosure. Process 400 as shown in FIG. 4 may be performed using one of GMDs 100, 150 or 160 as described above.

In an initial or periodic calibration step for a specific subject performed at any stage of process 400, the BCMD results may be compared to recent invasive blood test results, measured, for example, at different glucose levels of the subject. The invasive blood test results are converted to an illumination transmission model and the BCMD measurement results may then be calibrated to substantially match the invasive test results. Calibration may be performed by interaction with the BCMD controller or with an external device in data communication with the BCMD controller.

At step 402, the illumination source emits light at the chosen wavelengths. The different wavelengths may be emitted concurrently, sequentially, or in any other suitable manner (e.g., in batches). For example, in a case in which the detector is adapted for detecting multiple wavelengths (e.g., using a dedicated filter), the illumination source may emit substantially white light. It is noted that the term “white light” refers to light which include substantially equal levels of illumination across different wavelengths of a continuous part of the SWIR band (e.g., between 900 and 1400 nm). Other intensity distributions of a continuous part of the SWIR band may also be used, as well as narrower band light sources. Whenever the term “white light” is used in the disclosure, a broadband light that includes at least two separated wavelengths (or separated wavelength bands) which are measured by the detector may be used.

At step 404, for each wavelength, the detector may collect the reflected or transmitted signal from the skin at multiple distances from the illumination source.

At step 406, signal measurements collected at the same distance (d) from the source may be averaged by the processor so that the reflectance/transmittance data is dependent on the distance and the wavelength R(λ_i,d). In some implementations, the resulting measurements may be saved as a vector, as a graph, or as a function, etc.

At step 408, after collecting data for all wavelengths and for different distances from the light source, the data may be fitted to the calibration coefficients by the processor for DRS analysis. For example, this spectral response calibration produces a response difference function ΔI_{λ}(G) for each wavelength A and for each glucose concentration G. After calibration, the glucose concentration can be extracted from the inverse function to the response difference function. Thus, at step 410, the concentration of the measured compound (such as but not limited to glucose) may be provided, based on results of the calibration. Optionally, any combination of one or more of steps 406, 408, and 410 may be performed by at least one external device (external to the device in which detector array 112 is installed), based on the measurements of step 404.

Referring to stages 408 and 410, it is noted that the at least one processor which executes these stages (e.g., processor 114) may determine the concentration of glucose (or other material) in any one of several ways. For example, predetermined algorithms may be used, machine learning algorithms may be used, or any other suitable way. For example, stages 408 and/or 410 may include comparing a propagation model of light to different distances from the light source at different wavelengths to previously measured propagation models sampled at known levels of glucose. For example, stages 408 and/or 410 may include comparing the relative strengths of light of different wavelengths at discreet distances, and compare them to previously measured results, and so on.

Unless otherwise defined, all technical and scientific terms used herein have the same meaning as commonly understood by one of ordinary skill in the art. The materials, methods, and examples provided herein are illustrative only and not intended to be limiting.

As used herein the terms “machine learning” or “artificial intelligence” refer to use of algorithms on a computing device that parse data, learn from the data, and then make a determination or generate data, where the determination or generated data is not deterministically replicable (such as with deterministically oriented software as known in the art).

Implementation of the method and system of the present disclosure may involve performing or completing certain selected tasks or steps manually, automatically, or a combination thereof. Moreover, according to actual instrumentation and equipment of preferred embodiments of the method and system of the present disclosure, several selected steps may be implemented by hardware (HW) or by software (SW) on any operating system of any firmware, or by a combination thereof. For example, as hardware, selected steps of the disclosure could be implemented as a chip or a circuit. As software or algorithm, selected steps of the disclosure could be implemented as a plurality of software instructions being executed by a computer using any suitable operating system. In any case, selected steps of the method and system of the disclosure could be described as being performed by a data processor, such as a computing device for executing a plurality of instructions.

As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Although the present disclosure is described with regard to a “computing device”, a “computer”, or “mobile device”, it should be noted that optionally any device featuring a data processor and the ability to execute one or more instructions may be described as a computing device, including but not limited to any type of personal computer (PC), a server, a distributed server, a virtual server, a cloud computing platform, a cellular telephone, an IP telephone, a smartphone, a smart watch or a PDA (personal digital assistant). Any two or more of such devices in communication with each other may optionally comprise a “network” or a “computer network”.

To provide for interaction with a user, the systems and techniques described here can be implemented on a computer having a display device (a LED (light-emitting diode), or OLED (organic LED), or LCD (liquid crystal display) monitor/screen) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment or implementation are necessary in every embodiment or implementation. Further combinations of the above features and implementations are also considered to be within the scope of some embodiments or implementations.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. A device for detecting of compound levels in tissue, comprising:
an illumination source operable to emit light from an optical opening into the tissue;
a detector array having a plurality of photosites, each photosite operable to detect light of the illumination source travelling through the tissue, wherein different photosites of the detector array are located at different distances from the optical opening; and
a processor configured for determining the compound levels in the tissue based on differences in detected illumination levels at distinct wavelengths at different distances from the optical opening.

2. The device of claim 1, wherein the compound is glucose, wherein the light emitted from the illumination source has distinct wavelengths, wherein the distinct wavelengths are those where glucose is absorptive, and wherein the processor is adapted to determine glucose levels in the tissue based on the amount of light received by the detector array at each of the distinct wavelengths.

3. The device of claim 1, wherein the compound is hemoglobin, wherein the light emitted from the illumination source has distinct wavelengths, wherein the distinct wavelengths are those where hemoglobin is absorptive, and wherein the processor is configured to determine hemoglobin levels in the tissue based on the amount of light received by the detector array at each of the distinct wavelengths.

4. The device of claim 1, wherein the detector array comprises a color filter array (CFA) for permitting transmission of the distinct wavelengths to the detector array for measuring of the distinct wavelengths by the detector array.

5. The device of claim 4, wherein the CFA permits transmission of non-overlapping wavelengths to different rows or columns of the detector array.

6. The device of claim 1, wherein each photosite is adapted to detect one of the distinct wavelengths.

7. The device of claim 1, wherein the processor is configured to calibrate the device by comparing measured compound levels to an illumination transmission model determined for the subject based on an invasive measurement of different compound concentration levels.

8. The device of claim 1, wherein the processor is configured to determine an average of a plurality of detected illumination levels of different photosites whose distance from the illumination source is within a similar distance range.

9. The device of claim 1, wherein the illumination source comprises multiple illumination sources positioned in a ring arrangement.

10. A method for detecting compound levels in tissue, comprising: illuminating a surface of the tissue by an illumination source having an optical opening; detecting by a photodetector array comprising a plurality of photosites of reflected and/or transmitted light from the illumination source, wherein different photosites of the detector array are located at different distances from the optical opening; and determining compound levels in the tissue based on differences in detected illumination levels at distinct wavelengths at different distances from the optical opening by a processor.

11. The method of claim 10, wherein the compound is glucose, wherein the light emitted from the illumination source has distinct wave lengths and wherein the distinct wavelengths are those where glucose is absorptive.

12. The method of claim 11, further comprising determining by the processor of glucose levels in the tissue based on the amount of light received by the detector array at each of the distinct wavelengths.

13. The method of claim 10, wherein the detector array comprises a color filter array (CFA) for permitting transmission of the distinct wavelengths to the detector array for measuring of the distinct wavelengths by the detector array.

14. The method of claim 13, wherein the CFA permits transmission of non-overlapping wavelengths to each row or column of the detector array.

15. The method of claim 10, wherein each photosite is adapted to detect one of the distinct wavelengths.

16. The method of claim 10, further comprising calibration of the measurement by comparing measured compound levels to an illumination transmission model determined for the subject based on an invasive measurement of different glucose concentration levels.

17. The method of claim 10, further comprising determining by the processor of an average of a plurality of detected illumination levels of different photosites whose distance from the illumination source is within a similar distance range.

18. The method of claim 10, wherein the illumination source comprises multiple illumination sources wherein the multiple illumination sources are positioned in a ring arrangement.

19. A blood concentration measuring device comprising one or more processors and at least one non-transitory computer readable medium having stored thereon instructions that, when executed by the one or more processors, cause the blood concentration measuring device to: illuminate a surface of a tissue with an illumination source having an optical opening; detect reflected and/or transmitted light from the illumination source using a photodetector array comprising a plurality of photosites; and determine compound levels in the tissue based on differences in detected illumination levels at distinct wavelengths at different distances from the optical opening, wherein different photosites of the detector array are located at different distances from the optical opening.

20. The blood concentration measuring device of claim 19, wherein the compound is glucose, wherein the light emitted from the illumination source has distinct wavelengths and wherein the distinct wavelengths are those where glucose is absorptive.

21. The blood concentration measuring device of claim 20, storing instructions that, when executed by the one or more processors, cause the blood concentration measuring device to determine glucose levels in the tissue based on the amount of light received by the detector array at each of the distinct wavelengths.

22. The blood concentration measuring device of claim 19, wherein the detector array comprises a color filter array (CFA) for permitting transmission of the distinct wavelengths to the detector array for measuring of the distinct wavelengths by the detector array.

23. The blood concentration measuring device of claim 22, wherein the CFA permits transmission of non-overlapping wavelengths in each row or column of the detector array.

24. The blood concentration measuring device of claim 19, wherein each photosite is adapted to detect one of the distinct wavelengths.

25. The blood concentration measuring device of claim 19, storing instructions that, when executed by the one or more processors, cause the blood concentration measuring device to determine an average of a plurality of detected illumination levels of different photosites whose distance from the illumination source is within a similar distance range.
